(19) United States
(12) Patent Application Publication
(10) Pub. No.: US 2007/0135379 A1
(43) Pub. Date: Jun. 14, 2007

(54) Anhydrous pharmaceutical composition associating a siliconated agent and solubilised active principle

(75) Inventors: Claire Mallard, Mougins (FR); Eve Ferrara, Valbonne (FR)

Correspondence Address: OLIFF & BERRIDGE, PLC, P.O. BOX 19928, ALEXANDRIA, VA, 22320, US

(73) Assignee: GALDERMA RESEARCH & DEVELOPMENT, Biot (FR)

(21) Appl. No.: 10/591,864

(22) PCT Filed: Mar. 18, 2005
(86) PCT No.: PCT/FR05/50171

§ 371 Date: Dec. 5, 2006

(30) Foreign Application Priority Data
Mar. 22, 2004 (FR) 0402911

(51) Int. Cl. A61K 31/695; A61K 31/59

(52) U.S. Cl. 514/63; 514/167

(57) ABSTRACT

The invention relates to an anhydrous pharmaceutical composition associating at least one type of active principle and one type of siliconated agent which comprises at least one type of organopolysiloxane elastomer, wherein said active principle is embodied in the solubilised form thereof in said composition.

The present invention relates to stable, anhydrous pharmaceutical compositions combining at least one active ingredient and a silicone agent, said active ingredient being present in a solubilized form in said composition.

The present invention relates to the field of the formulation of an active ingredient with a view to pharmaceutical applications, in particular for topical application.

It is known that a certain number of compounds having an advantageous therapeutic activity are sensitive to oxidation and in particular undergo chemical degradation resulting in a substantial loss of their activity in the presence of water.

Consequently, it is advisable to formulate these active ingredients in compositions of anhydrous type.

The anhydrous compositions currently available, which make it possible to formulate water-sensitive active ingredients while at the same time ensuring that they have good chemical stability, are generally compositions of ointment type. These ointment-type compositions consist mainly of petroleum jelly, mineral oil and/or plant oil. However, these ointment-type compositions are not completely satisfactory. Some of them, after application, feel tacky and greasy, and are also shiny. In addition, generally, they are not always suited to the formulation of the active substance under consideration in a solubilized form.

Another alternative, in particular illustrated in documents EP 0 255 369 and U.S. Pat. No. 6,103,250, consists in providing formulations most commonly based on silicone derivatives in which the water-sensitive active substances are conditioned in a disperse form which is therefore generally prejudicial to optimal release and/or penetration of these active substances in the skin.

The aim of the present invention is precisely to provide an anhydrous pharmaceutical composition which makes it possible to overcome the abovementioned drawbacks.

More specifically, a subject of the present invention is an anhydrous pharmaceutical composition, in particular of gel type, combining at least one active ingredient and a silicone agent comprising at least one organopolysiloxane elastomer which does not comprise a hydrophilic group, said active ingredient being present in a solubilized form in said composition.

The term “solubilized form” is intended to mean a dispersion in the molecular state in a liquid, no crystallization of the active agent being visible to the naked eye or even under a cross-polarized optical microscope.

According to a second aspect, a subject of the present invention is the use of a silicone agent comprising at least one organopolysiloxane elastomer which does not comprise a hydrophilic group, for the preparation of an anhydrous pharmaceutical composition comprising at least one active ingredient in a solubilized form, and in particular with prolonged stability.

According to a third aspect of the invention, a subject of the latter is the use of an anhydrous pharmaceutical composition as defined above, for the manufacture of a medicament for use in the treatment of psoriasis.

Advantageously, the compositions according to the invention are found to be, after application, devoid of tacky, greasy and shiny effects, and, on the other hand, provide a soft feel. They are found to be particularly effective for preserving a satisfactory chemical stability of active ingredients sensitive to oxidation, to water and to aqueous media in general. In the compositions according to the invention, the active ingredients are in the solubilized state, which confers on the compositions better properties of release/penetration, in the skin, of the active ingredient, allied to more advantageous kinetics. It has also been noted that the compositions according to the invention exhibit a higher degree of release/penetration of the active ingredient, in the skin, than that obtained with a conventional ointment-type formulation.

For the purpose of the present invention, the expression “anhydrous composition” is intended to mean a composition substantially free of water, i.e. having a water content of less than or equal to 5%, and in particular of less than or equal to 3% by weight relative to the total weight of the composition, and preferably comprising no water. Compositions comprising a hydrophilic phase with a content greater than 10%, and also compositions of W/O or O/W emulsion type, sprays and other sprayable forms, are in particular excluded from the field of the invention.

For the purpose of the present invention, the expression “stable composition” is intended to mean a composition which does not exhibit any substantial modification of its macroscopic appearance over a period of at least three months when it is conserved at ambient temperature and at 40° C., and in which the content of intact active ingredient after three months at ambient temperature and at 40° C. is at least 70%, in particular at least 80%, more particularly at least 90%, or even at least 95% of the initial content by weight.

The expression “good release/penetration capacity” is intended to describe a better distribution of the composition and therefore of the active ingredient that it contains, through the stratum corneum of the skin and also through the subcutaneous layers such as the epidermis and the dermis.

The composition according to the invention is advantageously in the form of a gel.

Active Ingredient

As indicated above, the composition according to the invention comprises at least one active ingredient in a solubilized form in said composition.

The active ingredient considered is intended for pharmaceutical application, in particular dermatological application. It therefore has, in general, a therapeutic activity with respect to a dermatological ailment or to skin conditions.

The composition of the invention advantageously makes it possible, firstly, to satisfactorily formulate any active ingredient sensitive to oxidation, i.e. liable to be impaired by oxidation, and in particular impaired by the presence of water, and, secondly, to release said active ingredient in the layers of the skin.

Among the active ingredients which can be used in the compositions according to the invention, mention may in particular be made of vitamin D and its derivatives. The term “vitamin D” is intended to mean the various forms of vitamin D, such as, for example, vitamin D₂ or vitamin D₃. The term “vitamin D derivatives” is intended to mean compounds which exhibit biological properties similar to those of vitamin D, in particular properties of transactivation of vitamin D response elements (VDREs), such as an agonist or antagonist activity with respect to receptors of vitamin D or its derivatives. These compounds are generally not natural metabolites of vitamin D. They are in particular synthetic compounds comprising the backbone of vitamin D with modifications on the side chains and/or also comprising modifications in the backbone itself. Vitamin D-derived compounds which are useful according to the invention thus comprise structural analogs, for example bioaromatics.

By way of illustration of these vitamin D derivatives, mention may in particular be made of calcipotriol, calcitriol or 1,25-dihydroxyvitamin D₃, doxercalciferol, secalcitol, maxacalcitol, seocalcitol, tacalcitol, paricalcitol, falecalcitriol, 1α, 24S-dihydroxyvitamin D2,1 (S),3(R)-dihydroxy-20(R)-[((3-(2-hydroxy-2-propyl)phenyl)methoxy)methyl]-9,10-secopregna-5(Z),7(E),10(19)-triene, mixtures thereof and derivatives thereof.

As vitamin D derivatives which can be used according to the invention, mention may also be made of the derivatives described in WO 02/34235, WO 00/64450, EP1124779, EP1235824, EP1235777, WO 02/94754 and WO 03/050067.

According to a specific embodiment, the vitamin D derivatives used according to the invention are described in WO 00/26167. They are compounds which are structural analogs of vitamin D and which show a selective activity on cell proliferation and differentiation without having the characteristic of raising blood calcium levels.

These compounds can be represented by general formula (I) below: 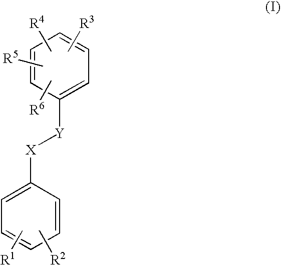
in which:
R¹ represents a hydrogen atom, a methyl radical or a -(CH₂)ₙ—OR⁷ radical,
R² represents a —(CH₂)ₙ—OR⁸ radical, n, R⁷ and R⁸ having the meanings given hereinafter,
X—Y represents a bond chosen from the bonds of formulae (a) to (d) below which can be read from left to right or vice versa: 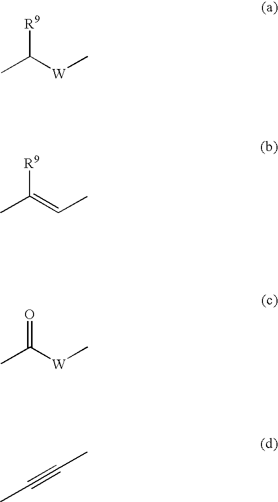
   R⁹ and W having the meanings given hereinafter,
R³ represents the chain of vitamin D₂ or of vitamin D₃, 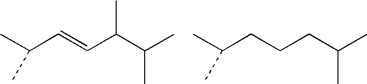
   the dashed lines representing the bond linking the chain to the benzene ring represented in figure (I), or
R³ represents a chain having from 4 to 8 carbon atoms, substituted with one or more hydroxyl groups, it being possible for the hydroxyl groups to be protected in acetoxy, methoxy, ethoxy, trimethylsilyloxy, tert-butyldimethylsilyloxy or tetrahydropyranyloxy form, and, optionally, in addition:
   substituted with one or more lower alkyl groups or cycloalkyl groups, and/or
   substituted with one or more halogen atoms, and/or
   substituted with one or more CF₃ groups, and/or
   in which one or more carbon atoms of the chain is (are) replaced with one or more oxygen, sulfur or nitrogen atoms, it being possible for the nitrogen atoms to be optionally substituted with lower alkyl radicals, and/or
   in which one or more single bonds of the chain are replaced with one or more double and/or triple bonds,
R³ being positioned on the benzene ring in the para or meta position with respect to the X—Y bond,
R^{4,} R⁵ and R⁶, which may be identical or different, represent a hydrogen atom, a lower alkyl radical, a halogen atom, an —OR¹⁰ radical or a polyether radical, R¹⁰ having the meaning given hereinafter,
n being 0, 1 or 2,
R⁷ and R⁸, which may be identical or different, represent a hydrogen atom, an acetyl radical, a trimethylsilyl radical, a tert-butyldimethylsilyl radical or a tetrahydropyranyl radical,
R⁹ representing a hydrogen atom or a lower alkyl radical,
W represents an oxygen or sulfur atom, a —CH₂— radical or a —NH— radical which can be optionally substituted with a lower alkyl radical,
R¹⁰ represents a hydrogen atom or a lower alkyl radical, and also the optical and geometric isomers of said compounds of formula (I) and the salts thereof when X—Y represent a bond of formula (a) and W represents an —NH— radical optionally substituted with a lower alkyl radical.

The expression “lower alkyl radical” is intended to mean a linear or branched alkyl radical having from 1 to 6 carbon atoms.

Among the compounds of formula (I) which can be used in the compositions of the present invention, mention may in particular be made of:
1. 6-[3-(3,4-bishydroxymethylbenzyloxy)phenyl]-2-methylhepta-3,5-dien-2-ol,
2. 7-[3-(3,4-bishydroxymethylphenoxymethyl)phenyl]-3-ethyloctan-3-ol,
3. 7-{3-[2-(3,4-bishydroxymethylphenyl)ethyl]phenyl}-3-ethylocta-4,6-dien-3-ol,
4. 6-{3-[2-(3,4-bishydroxymethylphenyl)ethyl]phenyl}-2-methylhepta-3,5-dien-2-ol,
5. 7-{3-[2-(3,4-bishydroxymethylphenyl)vinyl]phenyl}-3-ethylocta-4,6-dien-3-ol,
6. 7-[3-(3,4-bishydroxymethylbenzyloxy)phenyl]-3-ethyl-3-octanol,
7. 4E,6E)-7-[3-(3,4-bishydroxymethylbenzyloxy)phenyl]-3-ethylocta-4,6-dien-3-ol,
8. (4E,6E)-7-[3-(3,4-bishydroxymethylbenzyloxy)phenyl]-3-ethylnona-4,6-dien-3-ol,
9. (E)-7-[3-(3,4-bishydroxymethylbenzyloxy)phenyl]-3-ethyloct-4-en-3-ol,
10. (E)-7-[3-(3,4-bishydroxymethylbenzyloxy)phenyl]-3-ethyloct-6-en-3-ol,
11. (E)-7-[3-(3,4-bishydroxymethylbenzyloxy)phenyl]-3-ethyloct-6-en-4-yn-3-ol,
12. (4E,6E)-7-[3-(3,4-bishydroxymethylphenoxymethyl)phenyl]-3-ethylocta-4,6-dien-3-ol,
13. (E)-7-[3-(3,4-bishydroxymethylphenoxymethyl)phenyl]-3-ethylnon-6-en-3-ol,
14. (E)-7-{3-[(3,4-bishydroxymethylbenzyl)methylamino]phenyl}-3-ethyloct-6-en-3-ol, and
15. 7-[3-(3,4-bishydroxymethylbenzyloxy)phenyl]-3-ethyl-7-methyloctan-3-ol.

In particular, the pharmaceutical active ingredient incorporated into the composition according to the invention is (4E,6E)-7-[3-(3,4-bishydroxymethylbenzyloxy)phenyl]-3-ethylnona-4,6-dien-3-ol.

Vitamin D and its derivatives are generally used in dermatology in the treatment of psoriasis because they limit the excessive production of skin cells on affected surfaces and have proven advantages for the treatment of this condition which is characterized in particular by the presence of thick, squamous and dry lesions.

As active ingredients which can be used in the compositions according to the invention, mention may also be made of agents for modulating skin differentiation and/or proliferation and/or pigmentation, such as retinoic acid and its isomers, retinol and its esters, retinal, retinoids, estrogens, antibacterial agents, antibiotics, antiparasitic agents, antifungal agents, steroidal or nonsteroidal anti-inflammatories, anesthetics, antipruriginous agents, antiviral agents, keratolytic agents, free-radical scavengers, antiseborrheic agents, antidandruff agents, anti-acne agents, agents for combating hair loss, and vitamin C and its derivatives, with the proviso, as is indicated above, that the active agents are in a solubilized form in the composition according to the invention.

Advantageously, the composition according to the invention comprises from 0.0001% to 20% by weight relative to the total weight of the composition of an active agent, in particular from 0.01% to 15% by weight, and more particularly from 0.025% to 5% by weight.

Of course, the amount of active agent in the composition according to the invention will depend on the active agent under consideration.

Thus, when the active agent is chosen from vitamin D and its derivatives, the content of active agent is generally less than 2% by weight, in particular ranging from 0.01% to 0.5% by weight, and more particularly from 0.025% to 0.3% by weight.

According to a specific variant, the composition according to the invention comprises (4E,6E)-7-[3-(3,4-bishydroxymethylbenzyloxy)phenyl]-3-ethylnona-4,6-dien-3-ol at the concentration of 0.3% by weight.

Solvent Agent

The pharmaceutical composition according to the invention generally comprises at least one solvent agent or mixture for the active ingredient.

This solvent agent is chosen from pharmaceutically acceptable compounds, i.e. compounds the use of which is in particular compatible with use on the skin, the mucous membranes and/or the keratin fibers.

It is generally fluid, and in particular liquid, at ambient temperature and atmospheric pressure.

Particularly suitable as solvent agents according to the invention are solvents of alcoholic type, and more particularly aliphatic alcohols containing one to six carbon atoms, chosen from methanol, ethanol, isopropanol and butanol, and mixtures thereof.

As a solvent agent which can be used in the compositions according to the invention and which is suitable in particular for the solubilization of vitamin D derivatives, mention may also be made of a compound chosen from the group consisting of:

(i) compounds of general formula (II): R¹³(OCH₂C(R¹¹)H)ₓOR¹² (II) in which x is an integer ranging from 2 to 60, R¹¹ in each of the units x is independently H or CH₃, R¹² is a linear or branched C₁₋₂₀ alkyl or a benzoyl radical, and R¹³ is H or a phenylcarbonyloxy radical;

(ii) di(C₄₋₁₀ linear or branched alkyl) C₄₋₈ dicarboxylic acid esters, and

(iii) linear or branched C₁₂₋₁₈ alkyl benzoates.

Of course, the choice of the solvent agent depends in particular on the active ingredient to be solubilized.

The solvent agent is more particularly absolute ethanol, in particular when the active ingredient to be solubilized is vitamin D or one of its derivatives.

The solvent agent for the active ingredient as defined above is generally present in the compositions according to the invention in an amount, firstly, sufficient to provide the required solubility of the active ingredient to be formulated and, secondly, compatible with the need to preserve prolonged chemical stability of this same active ingredient. In other words, the solvent agent must be chemically inert with respect to the active ingredient.

The presence of this solvent agent can also be useful for promoting the compatibility of the silicone agent with other component(s) of the composition, for instance of hydrocarbon-based compound type, such as waxes. The ethanol is thus most particularly useful in the case of a silicone agent-wax mixture.

For example, it may be present at a content of from 1% to 50%, in particular from 2% to 40%, and more particularly from 5% to 10% by weight relative to the total weight of the composition.

According to a specific embodiment of the invention, the composition comprises, as active agent, a vitamin D derivative in a solubilized form, and absolute ethanol at a content of from 1% to 50%, in particular from 2% to 40%, and more particularly from 5% to 10% by weight relative to the total weight of the composition.

The solvent agent, in the composition according to the invention, also confers a beneficial effect on the degree of penetration into the skin of the active ingredient as defined hereinafter.

Silicone Agent

The composition according to the invention comprises at least one silicone agent.

In general, this silicone agent comprises at least one organopolysiloxane elastomer.

The expression “organopolysiloxane elastomer” denotes, in its most general definition, any chemically crosslinked siloxane polymer which exhibits viscoelastic properties.

The term “viscoelastic properties” is intended to mean the ability of the elastomer to distort to a certain point, when subjected to a mechanical load, and to return to its original shape following the removal of said load.

The organopolysiloxane elastomers in accordance with the invention do not contain a hydrophilic group. The term “hydrophilic group”, according to the invention, is intended to mean for example a polyoxyalkylene-type group or a glycol-type group.

The silicone agent defined above can exercise in particular the function of thickening agent in the compositions according to the invention. It can also participate in the stabilization thereof.

Organopolysiloxane elastomers which can be used in the compositions according to the invention are in particular described in patents U.S. Pat. No. 4,980,167 and U.S. Pat, No. 4,742,142. They may in particular be compounds resulting from addition reactions, i.e. products of hydrosilylation or products of addition polymerization of an organopolysiloxane having unsaturated groups such as vinyl or allyl groups, in particular linked to at least one terminal Si atom and of another silicone compound capable of participating in the addition reaction, such as an organohydrogenopolysiloxane.

The content of organopolysiloxane elastomer in the compositions according to the invention can vary substantially, in particular according to the desired viscosity of the composition, and also as a function of the optional presence of an additional thickening agent. The optimal content as a function of these various parameters can be readily determined by those skilled in the art using simple routine experiments.

In general, the content of organopolysiloxane elastomer in the compositions according to the invention is from 1% to 20%, in particular from 4% to 12%, and more particularly from 5% to 10% by weight relative to the total weight of the composition.

According to a specific embodiment, the organopolysiloxane elastomer is formulated in a vehicle comprising at least one volatile silicone oil.

For the purpose of the invention, the term “volatile compound” is intended to mean any compound capable of evaporating on contact with the skin, the mucous membranes or the keratin fibers in less than one hour, at ambient temperature and atmospheric pressure. The volatile compound is a pharmaceutically acceptable volatile compound which is liquid at ambient temperature, having in particular a non-zero vapor pressure, at ambient temperature and at atmospheric pressure, in particular having a vapor pressure ranging from 0.13 Pa to 40 000 Pa (10⁻³ to 300 mm Hg), in particular ranging from 1.3 Pa to 13 000 Pa (0.01 to 100 mm Hg), and more particularly ranging from 1.3 Pa to 1300 Pa (0.01 to 10 mm Hg).

As volatile silicone oils, use may, for example, be made of volatile linear or cyclic polyorganosiloxane oils, in particular those having a viscosity ≧6 centistokes (6×10⁻⁶ m²/s), and having in particular from 2 to 10 silicon atoms, these silicones optionally containing alkyl or alkoxy groups having from 1 to 22 carbon atoms. The volatile silicone oils include in particular cyclomethicones and dimethicones of low molecular weight, or mixtures thereof. In particular, the volatile silicone oils are chosen from methylated cyclic organopolysiloxanes having ring sizes ranging from 4 to 12, such as octamethylcyclotetrasiloxane and decamethylcyclopentasiloxane. As a volatile silicone oil which can be used in the invention, mention may also be made, in particular, of dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

According to a specific embodiment of the invention, the silicone agent used in the preparation of the compositions according to the invention is provided in the form of an organopolysiloxane elastomer as defined above and formulated in a proportion of from 1% to 30%, and in particular from 10% to 20% by weight relative to the total weight of said silicone agent, in at least one volatile silicone oil as defined above.

Among the organopolysiloxane elastomers which can be used in the compositions according to the invention, mention may be made of those prepared by crosslinking reaction between polysiloxanes (A) containing ≡Si—H groups as defined below, an alpha,omega-diene (B) in the presence of a catalyst, and a low molecular weight linear or cyclic polysiloxane (C).

The polysiloxane (A) containing the ≡Si—H unit can be represented by the compounds of formula R₃¹⁴SiO(R¹⁵₂SiO)ₐ(R¹⁶HSiO)_{b}SiR₃¹⁴ here denoted as type A¹, and compounds of formula HR₂¹⁴SiO(R¹⁵₂SiO)_{c}SiR₂¹⁴H or of formula HR₂¹⁴SiO(R¹⁶₂SiO)ₐ(R¹⁶HSiO)_{b}SiR₂¹⁴H here denoted as type A². In these formulae, R¹⁴, R¹⁵ and R¹⁶ are alkyl groups having from 1 to 6 carbon atoms, a is an integer ranging from 0 to 250, b is an integer ranging from 1 to 250, and c is an integer ranging from 0 to 250. The molar ratio of the compounds A²:A¹ is from 0 to 20, in particular from 0 to 5.

The alpha,omega-diene (B) is a compound of formula CH₂—CH(CH₂)_{d}CH—CH₂ in which d is an integer ranging from 1 to 20. Representative examples of suitable alpha,omega-dienes are 1,4-pentadiene, 1,5-hexadiene, 1,6-heptadiene, 1,7-octadiene, 1,8-nonadiene, 1,9-decadiene, 1,11-dodecadiene, 1,13-tetradecadiene and 1,19-eicosadiene.

The expression “low molecular weight polysiloxane (C)” encompasses (i) linear or cyclic, volatile, low molecular weight methylsiloxanes, (ii) volatile or nonvolatile, linear or cyclic, low molecular weight alkylsiloxanes and arylsiloxanes, and (iii) linear or cyclic, low molecular weight functional siloxanes. Advantageously, the oil (C) is chosen from linear or cyclic, volatile low molecular weight methylsiloxanes.

As volatile methylsiloxanes, mention may in particular be made of linear volatile methylsiloxanes, such as hexamethyldisiloxane, octamethyltrisiloxane, decamethyltetrasiloxane, dodecamethylpentasiloxane, tetradecamethylhexasiloxane and hexadecamethylheptasiloxane.

As cyclic volatile methylsiloxanes, mention may in particular be made of hexamethylcyclotrisiloxane, octamethylcyclotetrasiloxane, decamethylcyclopentasiloxane and dodecamethylcyclohexasiloxane,

As branched volatile methylsiloxanes, mention may in particular be made of heptamethyl-3-[(trimethylsilyl)oxy]trisiloxane, hexamethyl-3,3-bis[(trimethylsilyl)oxy]-trisiloxane and pentamethyl[(trimethylsilyl)oxy]cyclotrisiloxane.

As indicated above, also suitable in the present invention are nonvolatile low molecular weight polysiloxanes (C) such as those corresponding to the general formula

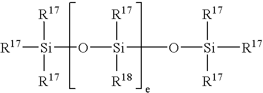

in which e is such that the polymers corresponding to this formula have a viscosity in the range of from approximately 100 to 1000 centistokes (mm²/sec).

R¹⁷ and R¹⁸ are alkyl radicals having from 1 to 20 carbon atoms or an aryl group such as a phenyl group. In particular, e is chosen in the range of from 80 to 375.

Among these low molecular weight polysiloxanes (C), mention may in particular be made of polydimethylsiloxane, polydiethylsiloxane, polymethylethylsiloxane, polymethylphenylsiloxane and polydiphenylsiloxane.

Functionalized low molecular weight polysiloxanes (C) can be represented by fluid siloxanes bearing acrylamide, acrylate, amide, amino, carbinol, carboxyl, chloroalkyl, epoxy, glycol, cetal, mercapto, methyl ester, perfluoro and silanol functions.

Organopolysiloxane elastomers resulting from the crosslinking reaction described above are in particular described in patent U.S. Pat. No. 5,654,362.

Among the organopolysiloxane elastomers preferentially used in the compositions according to the invention, mention may in particular be made of the elastomers described in patent U.S. Pat. No. 5,929,164.

Described in particular is the organopolysiloxane elastomer used most preferentially according to the invention, the “ST Elastomer 10®” from DOW CORNING, which is an organopolysiloxane elastomer formulated in a decamethylcyclopentasiloxane oil provided in the form of a thick and translucent gel.

This type of organopolysiloxane elastomer is synthesized by means of a crosslinking reaction similar to that described above, i.e. prepared by means of a crosslinking reaction between polysiloxanes (A) containing ≡Si—H groups as defined above, an alpha,omega-diene (B) in the presence of a catalyst, and a high molecular weight linear or cyclic polysiloxane (C) to which vinylsiloxanes (or vinylsilanes) (A′) containing —CH═CH₂ vinyl groups are added.

In fact, it has been demonstrated that the addition of these vinylsiloxanes (or vinylsilanes) blocks the remaining SiH functions which are not reacted (“quenching agent”). The compounds (A′) which can be used for the preparation of the preferred silicone agents according to the invention are such as those described in application U.S. Pat. No. 5,929,164. By way of examples of such vinylsiloxane or vinylsilane compounds (A′), mention may be made of vinyl-t-butyldimethylsilane, vinyldiethylmethylsilane, vinylethyldimethylsilane, vinyltriethylsilane, vinyltrimethylsilane, divinyldimethylsilane, divinyltetramethyldisilane, vinylpentamethyldisiloxane, 1,3-divinyltetramethyldisiloxane, a vinyltrisiloxane of structure (CH₃)₃SiOSi(CH═CH₂)(CH₃)OSi(CH₃)₃, 1,5-divinylhexamethyltrisiloxane, and a divinylsiloxane oligomer having a structure (CH₂═CH)Me₂SiO(Me₂SiO)₈SiMe₂(CH═CH₂).

The alpha,omega-diene (B) preferred according to any one of the crosslinking reactions described above is 1,5-hexadiene.

Also suitable as organopolysiloxane elastomers in accordance with the invention are silicone polymers having an average molecular weight of at least 10 000 (for example ranging from 10 000 to 10 000 000). Examples of silicone polymers include crosslinked siloxane copolymers, for example of dimethicone or of dimethicone derivatives, such as stearyl methyl dimethyl siloxane copolymer (“Gransil SR-CYC®” from the company Grant Industries), “Polysilicone-11®” (i.e. a crosslinked silicone elastomer formed by the reaction of silicone comprising a vinyl ending and of methylhydrodimethylsiloxane in the presence of cyclomethicone), cetearyl dimethicone/vinyl dimethicone crosslinked copolymers (i.e. a copolymer of cetearyl dimethicone crosslinked with a vinyldimethylpolysiloxane), a crosslinked polymer of dimethicone/phenyl vinyl dimethicone (i.e. a copolymer of dimethylpolysiloxane crosslinked with phenylvinyldimethylsiloxane), and a crosslinked copolymer of dimethicone/vinyl dimethicone (i.e. a copolymer of dimethylpolysiloxane crosslinked with vinyldimethylsiloxane).

Such organopolysiloxane elastomers, in the form of a gel, can be obtained commercially in particular from Grant Industries. Examples of such organopolysiloxane elastomers comprise the mixtures of cyclomethicone and polysilicone-11, for example, sold under the name “Gransil GCM®”, of cyclotetrasiloxane, petroleum jelly and polysilicone-11, for example, sold under the name “Gransil PS-4®”, of cyclopentasiloxane, petroleum jelly and polysilicone-11, for example, sold under the name “Gransil PS-5®”, of cyclopentasiloxane, dimethicone and polysilicone-11, for example, sold under the name “Gransil DMCM-5®”, of cyclotetrasiloxane, dimethicone and polysilicone-11, for example, sold under the name “Gransil DMCM-4®”, of polysilicone-11 and isododecane, for example, sold under the name “Gransil IDS®”, and of cyclomethicone, polysilicone-11, petroleum jelly and phytosphingosine, for example, sold under the name “Gransil SPH®”. Examples of gels available from the company “General Electric” are in particular a crosslinked polymer of cyclopentasiloxane and dimethicone/vinyl dimethicone crosslinked polymer “SFE839®”.

Other organopolysiloxane elastomers can also be obtained commercially, in particular from Shin-Etsu under the following references: KSG-15, KSG-16, KSG-17 and KSG-21.

The silicone agent is generally present in the compositions according to the invention at a content of from 20% to 80%, in particular from 30% to 70%, and more particularly from 40% to 65% by weight expressed as total weight of the silicone agent relative to the total weight of the composition.

By way of illustration of the compositions in accordance with the present invention, mention may more particularly be made of anhydrous pharmaceutical compositions, in particular of gel type comprising at least one silicone agent, a hydrocarbon-based compound, in particular of pasty or solid type, such as, for example, a wax, an active ingredient in a solubilized form, in particular vitamin D or one of its derivatives, and a solvent of alcoholic type, and in particular absolute ethanol.

Other Ingredients

The composition according to the invention can also comprise various other ingredients. Of course, the choice of these additional ingredients, just as that of the respective amounts thereof, is made in such a way as not to be detrimental to the properties expected for the composition. In other words, these compounds must not affect the chemical stability of the associated active ingredient, nor the solubility thereof

Additional Thickening Agent

The composition according to the invention can thus also comprise at least one additional thickening agent different from the silicone agent as defined above.

The additional thickening agent can be pasty or solid at ambient temperature, like, for example, a pasty or solid hydrocarbon-based compound like a wax.

The term “wax” is intended to mean, in general, a lipophilic compound which is solid at ambient temperature (25° C.), which has a reversible solid/liquid state change, and which has a melting point greater than or equal to 30° C., possibly ranging up to 200° C., and in particular up to 120° C.

The waxes which can be used in the compositions according to the invention can be of animal, plant, mineral or synthetic origin, and mixtures thereof.

Surprisingly, it is also possible to use, as additional thickening agent, hydrocarbon-based compounds, and in particular waxes, which are well known to be relatively incompatible with silicone compounds, while at the same time conserving a stable composition.

According to a specific embodiment, the hydrocarbon-based wax can be chosen from glyceryl esters of saturated and unsaturated fatty acids, in particular polyunsaturated fatty acids, having in particular from 10 to 24 carbon atoms, and unsaturated fatty acids, and in particular from polyunsaturated fatty acids.

As hydrocarbon-based waxes of polyunsaturated fatty acid glyceryl ester type which can be used in the compositions according to the invention, mention may in particular be made of the (C₁₆-C₁₈) atomized glyceryl dipalmitostearate sold under the name “Precirol ATO 5®” by the company GATTEFOSSE, (C₂₂) atomized glyceryl behenate, for example, sold under the name “Compritol®” by the company GATTEFOSSE, and mixtures thereof.

Use may also be made of hydrocarbon-based waxes such as beeswax, lanolin wax, and Chinese insect waxes; rice wax, carnauba wax, candelilla wax, ouricury wax, alfalfa wax, cork fiber wax, sugarcane wax, Japan wax and sumach wax; montan wax, microcrystalline waxes, paraffins and ozokerite; polyethylene waxes, waxes obtained by Fisher-Tropsch synthesis and waxy copolymers, and also esters thereof.

Mention may also be made of waxes obtained by catalytic hydrogenation of animal or plant oils containing linear or branched C₈-C₃₂ fatty chains. Among these, mention may in particular be made of hydrogenated jojoba oil, isomerized jojoba oil such as the trans-isomerized partially hydrogenated jojoba oil manufactured or sold by the company Desert Whale under the commercial reference ISO-JOJOBA-50®, hydrogenated sunflower oil, hydrogenated castor oil, hydrogenated coconut oil, hydrogenated lanolin oil, bis(1,1,1-trimethylolpropane) tetrastearate sold under the name “HEST 2T-4S” by the company HETERENE, and bis(1,1,1-trimethylolpropane) tetrabehenate sold under the name HEST 2T-4B by the company HETERENE.

Mention may also be made of silicone waxes and fluoro waxes.

Use may also be made of the wax obtained by hydrogenation of olive oil esterified with stearyl alcohol sold under the name “PHYTOWAX Olive 18 L 57” or else the waxes obtained by hydrogenation of castor oil esterified with cetyl alcohol sold under the name “PHYTOWAX ricin 16L64 and 22L73”, by the company SOPHIM. Such waxes are described in application FR-A-2792190.

The content of additional thickening agent depends of course on the desired viscosity of the composition, and on the content of silicone thickening agent. The content can be determined by those skilled in the art by means of simple routine manipulation.

According to a specific embodiment, the use of additional thickening agent as defined above in suitable proportions can also make it possible to confer an occlusive nature on the composition according to the invention. Advantageously, these occlusive-type compositions facilitate most particularly the release of the active ingredient.

The term “occlusive nature” is intended to mean the ability of the composition to retain water, i.e. to limit the imperceptible loss of water from the skin after application. Such a composition makes it possible to maintain the moisturization of the skin while avoiding or decreasing the evaporation of water through the skin.

In general, the content of additional thickening agent, and in particular of pasty or solid hydrocarbon-based compound, is from 2% to 80%, in particular from 4% to 30%, and more particularly from 6% to 20% by weight relative to the total weight of the composition.

Agent for Diluting the Silicone Agent

The composition according to the invention can also comprise at least one agent for diluting the silicone agent, and in particular an agent for diluting the organopolysiloxane elastomer.

Among the diluting agents which can be used in the compositions, mention may in particular be made of the linear or cyclic, volatile silicone oils as defined above.

In particular, when the organopolysiloxane elastomer is formulated in a vehicle, the diluting agent can be chosen from the compounds forming this vehicle.

As diluting agent which can be used in the compositions according to the invention, mention may in particular be made of decamethylcyclopentasiloxane such as that sold under the name “Mirasil CM5®” by the company RHODIA or under the name “ST-Cyclomethicone 5-NF®” by the company DOW CORNING.

Here again, the amount of diluting agent introduced during the preparation of the composition according to the invention depends of course on the desired viscosity of the composition. The amount to be introduced can be determined by those skilled in the art by means of simple routine experiments.

Advantageously, the diluting agent used in the compositions according to the invention is chosen from cyclic volatile silicones.

In general, the total content of agent for diluting the organopolysiloxane elastomer, and more particularly of cyclic or linear, volatile or nonvolatile, silicone oil, is from 10% to 70%, in particular from 20% to 50%, and more particularly from 25% to 40% by weight relative to the total weight of the composition.

Agent for Promoting Penetration of the Active Ingredient

The composition according to the invention can also comprise at least one agent for promoting penetration of the active ingredient into the skin.

Such agents can also be solvents for the active ingredient and can be chosen from the compounds mentioned as such above.

Particularly suitable as propenetrating agents according to the invention are glycols such as those having from 2 to 8 carbon atoms, for instance, in particular, propylene glycol, ethylene glycol, 1,3-butylene glycol and dipropylene glycol, of glycerol type, glycol ethers such as methyl glycol, 2-ethoxyethyl acetate, 2-methoxyethyl acetate, and in particular diethylene glycol monoethyl ether, in particular that sold under the name “Transcutol P®” by the company GATTEFOSSE, and mixtures thereof.

Particularly suitable for the invention, as propenetrating agents, are glycol ethers, fatty acids, fatty acid esters, glycol esters, glycerides, azones, polysorbates, alkanols, dimethyl sulfoxide, and mixtures thereof. Mention may in particular be made of oleyl alcohol, oleic acid, the azone laurocapram or 1-n-dodecylazacycloheptan-2-one, mono- and diester of propylene glycol and of fat and of fatty acids such as, for example, propylene glycol monocaprylate and propylene glycol monolaurate, triglycerides and lipids such as linoleic acid, macrogol glycerides or glycerides of propylene glycol and of fatty acids, for example stearoyl macrogol glycerides, oleoyl macrogol glycerides, lauroyl macrogol glycerides, oleoyl macrogol-6-glycerides and lauroyl macrogol-6-glycerides, fatty acid esters of polyethylene glycol and of a glyceride, for example caprylocaproyl macrogol glycerides, capryl-caproyl macrogol glycerides, oleoyl macrogol glycerides, polyoxyl 40 hydrogenated castor oil sold under the name “Cremophore RH 40”, polysorbate 80 sold under the name “Tween 80”, dodecyl azacycloheptanone, and mixtures thereof.

The content of agent(s) for promoting penetration into the skin as defined above is generally from 2% to 30%, in particular from 4% to 25%, and more particularly from 5% to 15% by weight relative to the total weight of the composition.

Additional Additives

Among the pharmaceutically acceptable additives which can be introduced into the compositions according to the invention, mention may in particular be made of compounds of nonvolatile oil type generally having a viscosity greater than approximately 10 centipoises at 25° C. and which can have a viscosity ranging up to 1 000 000 centipoises at 25° C.; mention may in particular be made of nonvolatile hydrocarbon-based oils, glyceryl esters of fatty acids, and fatty acid glycerides.

As a nonvolatile hydrocarbon-based oil, mention may in particular be made of:
hydrocarbon-based oils of plant origin, such as triglycerides consisting of fatty acid esters of glycerol, the fatty acids of which can have varied chain lengths of C₄ to C₂₄, it being possible for the latter to be linear or branched, and saturated or unsaturated; these oils are in particular wheatgerm oil, sunflower oil, grapeseed oil, sesame oil, maize oil, apricot oil, castor oil, shea oil, avocado oil, olive oil, soya oil, sweet almond oil, palm oil, rapeseed oil, cottonseed oil, hazelnut oil, macadamia oil, jojoba oil, alfalfa oil, poppy seed oil, pumpkin oil, sesame oil, marrow oil, rapeseed oil, blackcurrant oil, evening primrose oil, millet oil, barley oil, quinoa oil, rye oil, safflower oil, candlenut oil, passionflower oil, musk rose oil; or else triglycerides of caprylic/capric acids such as those sold by the company STEARINERIES DUBOIS or those sold under the names “Miglyol 810®”, “812®” and “818®” by the company DYNAMIT NOBEL, lanolin oil, triisocetyl citrate, C₁₀-C₁₈ triglycerides, caprylic/capric triglycerides;
synthetic ethers having from 10 to 40 carbon atoms;
linear or branched hydrocarbons of mineral or synthetic origin, such as petroleum jelly, polydecenes, hydrogenated polyisobutene such as parleam, squalane, and mixtures thereof;
synthetic esters such as oils of formula R¹⁹COOR²⁰ in which R¹⁹ represents the residue of a linear or branched fatty acid containing from 1 to 40 carbon atoms and R²⁰ represents a hydrocarbon-based chain that is in particular branched, containing from 1 to 40 carbon atoms, provided that R¹⁹+R²⁰≧10, for instance purcellin oil (cetylstearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, alcohol or polyalcohol octanoates, decanoates or ricinoleates, such as propylene glycol dioctanoate; hydroxylated esters, such as isostearyl lactate or diisostearyl malate; and pentaerythritol esters; and mixtures thereof; mention may also be made, for example, of nonvolatile oils of formula R²¹CO—OR²² in which R²¹ and R²² each independently represent a linear or branched alkyl radical, or a C₁ to C₂₅, in particular C₄ to C₂₀, alkenyl, alkoxycarbonylalkyl or alkylcarbonyloxyalkyl radical. Examples of such esters encompass isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco caprate/dicaprylate, decyl isostearate, isodecyl oleate, isodecyl neopentanoate, isohexyl neopentanoate, octyl palmitate, dioctyl malate, tridecyl octanoate, myristyl myristate and octododecanol;
fatty alcohols which are liquid at ambient temperature and contain a branched and/or unsaturated carbon chain having from 12 to 26 carbon atoms, such as octyldodecanol, isostearyl alcohol, oleyl alcohol, 2-hexyldecanol, 2-butyloctanol, 2-undecylpentadecanol;
higher fatty acids such as oleic acid, linoleic acid or linolenic acid; and mixtures thereof.

As fatty acid glycerides, mention may also be made of synthetic or semisynthetic compounds such as mono-, di- and triglycerides of fatty acids which are natural oils or fats which have been modified, for example glyceryl stearate, glyceryl dioleate, glyceryl distearate, glyceryl trioctanoate, glyceryl linoleate, glyceryl myristate, glyceryl isostearate, PEG castor oils, PEG glyceryl oleates, PEG glyceryl stearates, etc.

Also suitable in the present invention are nonvolatile hydrocarbon-based oils such as isoparaffins, mineral oils, etc.

The compositions according to the invention can also comprise at least one additional additive. Among these additives, mention may in particular be made of antioxidants, dyes, surfactants, fragrances, lipophilic sunscreens, etc.

Advantageously, the compositions according to the invention can be free of preserving system given their essentially anhydrous nature and the presence of the silicone agent which is relatively unfavorable to microbial development.

According to a specific embodiment of the invention, the composition is free of antiperspirant compound, in particular such as astringent metal salts. The composition according to the invention is in particular free of mineral or organic salts of aluminum, of zirconium and/or of zinc.

The composition according to the invention can also be free of particulate material, in particular of particulate pigment and/or filler, such as, for example, free of particles of mica or mica derivatives or of silica or silica derivatives.

The composition according to the invention can be of nonocclusive type, or else of occlusive type, in particular when it comprises an additional thickening agent.

The composition according to the invention can be transparent, translucent or opaque. It can be colored or colorless.

The composition is generally conserved in a watertight/airtight packaging, where appropriate equipped with a moisture-absorbing device.

It can be administered topically, with a periodicity which can be two or three applications a day.

The composition according to the invention is generally prepared by mixing at least two distinct phases: a phase comprising at least the silicone agent and a phase comprising at least the active ingredient and the solvent agent or mixture for said active ingredient. Where appropriate, the composition to be prepared also comprises fatty additives. In such a case, a third phase combining these fatty additives is prepared separately.

A subject of the present invention is also the use of a composition according to the invention, for the manufacture of a medicament for use in the treatment:
of dermatological conditions linked to a keratinization disorder related to differentiation and proliferation, in particular common acne, comedone acne, polymorphic acne, acne rosacea, nodulocystic acne, acne conglobata, senile acne, secondary acne such as solar acne, acne medicamentosa or occupational acne,
of ichtyosis, ichtyosiform states, Darrier's disease, palmoplantar keratoderma, leukoplakia and leukoplakiform states, cutaneous or mucosal (buccal) lichen,
of dermatological conditions with an inflammatory immunoallergic component, with or without cell proliferation disorder, and in particular cutaneous, mucosal or ungual psoriasis, psoriatic rheumatism, or cutaneous atopy, such as eczema, respiratory atopy or gingival hypertrophy,
of benign or malignant, dermal or epidermal proliferations of viral or nonviral origin, in particular verruca vulgaris, verruca plana, epidermodysplasia verruciformis, oral or florid papillomatoses, T lymphoma,
of proliferations which may be induced by ultraviolet radiation, in particular baso- and spinocellular epitheliomas,
of precancerous skin lesions, in particular keratoacanthomas,
of immune dermatoses, in particular lupus erythematosus,
of bullous immune diseases,
of collagen diseases, in particular scleroderma,
of dermatological or general conditions with an immunological component,
of skin disorders due to exposure to UV radiation, photoinduced or chronological skin aging or actinic keratoses and pigmentations, or any pathologies associated with chronological or actinic aging, in particular xerosis,
of sebaceous function disorders, in particular acne hyperseborrhea, simple seborrhea or seborrheic dermatitis,
of cicatrization disorders or stretch marks,
of pigmentation disorders, such as hyperpigmentation, melasma, hypopigmentation or vitiligo,
of lipid metabolism conditions, such as obesity, hyperlipidemia, non-insulin-dependent diabetes or syndrome X,
of inflammatory conditions such as arthritis,
of cancerous or precancerous states,
of alopecia of various origins, in particular alopecia due to chemotherapy or to radiation,
of immune system disorders, such as asthma, diabetes mellitus type I, multiple sclerosis, or other selective dysfunctions of the immune system, or
of conditions of the cardiovascular system, such as arteriosclerosis or hypertension.

More particularly, a subject of the present invention is the use of a composition according to the invention, for the manufacture of a medicament for use in the treatment of psoriasis.

The examples which follow are given by way of nonlimiting illustration of the invention.

### EXAMPLES 1 TO 5

According to the procedure described hereinafter, the compositions presented in table 1 below were prepared (in this table, the amounts indicated are as a percentage by weight and are expressed relative to the total weight of the composition):

TABLE 1

| | Example 1 | Example 2 | Example 3 | Example 4 | Example 5 |
|---|---|---|---|---|---|
| Phase 1 | | | | | |
| (C₁₆-C₁₈) atomized glyceryl | 8.0 | 8.0 | 8.0 | 8.0 | — |
| dipalmitostearate (“Precirol | | | | | |
| ATO 5 ®” from GATTEFOSSE) | | | | | |
| (C₂₂) atomized glyceryl | — | — | — | — | 8.0 |
| behenate (“Compritol ®” from | | | | | |
| GATTEFOSSE) | | | | | |
| Isopropyl myristate | 10.0 | 10.0 | 10.0 | 10.0 | 10.0 |
| Propylene glycol (from MERCK) | — | — | 5.0 | 5.0 | — |
| Phase 2 | | | | | |
| Purified diethylene glycol | 5.0 | 5.0 | — | — | 5.0 |
| monoethyl ether (“Transcutol P ®” | | | | | |
| from GATTEFOSSE) | | | | | |
| Mixture of silicone elastomer and | 45.0 | 44.8 | 45.0 | 44.8 | 40.0 |
| of decamethylcyclopentasiloxane | | | | | |
| (“ST-Elastomer 10 ®” from DOW | | | | | |
| CORNING) | | | | | |
| Decamethylcyclopentasiloxane | 26.9 | 26.9 | 26.9 | 26.9 | 31.7 |
| (“Mirasil CM5 ®” from RHODIA) | | | | | |
| Phase 3 | | | | | |
| Absolute ethanol | 5.0 | 5.0 | 5.0 | 5.0 | 5.0 |
| Vitamin D derivative: | 0.10 | 0.30 | 0.10 | 0.30 | 0.30 |
| (4E,6E)-7-[3-(3,4-bishydroxy- | | | | | |
| methylbenzyloxy)phenyl]-3-ethyl- | | | | | |
| nona-4,6-dien-3-ol | | | | | |

Methods for Preparing the Compositions of Examples 1 to 5

All the manipulations involving the vitamin D derivative under consideration are carried out under inactinic light.

Preparation of Phase 1

The ingredients of phase 1 as defined in table 1 above are introduced into a 600 ml glass beaker, which is then heated in a waterbath to a temperature at least 10° C. above the melting point of the wax used, i.e. to a temperature of the order of 65° C. when the composition to be prepared comprises glyceryl dipalmitostearate, and of the order of 80° C. when the composition to be prepared comprises glyceryl behenate.

Preparation of Phase 2

The ingredients of phase 2 as defined in table 1 above (with the exception of the diethylene glycol monoethyl ether) are introduced into a 500 ml beaker, and are then mixed by stirring with a Rayneri blender equipped with a deflocculating paddle, the beaker being covered with aluminum foil in order to minimize the volatilization of the silicone oil. The mixture is homogenized at moderate speed until a transparent gel that is more fluid than initially is obtained. The stirring is then stopped and the mixture is rapidly heated to 60° C. in a waterbath.

Preparation of Phase 3

The ethanol and then the vitamin D derivative are introduced into a 30 ml glass vial containing a magnetic bar. After having stoppered the vial, the latter is placed on a magnetic stirrer plate, at a sufficient stirring speed to obtain a vortex, until the vitamin D derivative is solubilized.

Procedure

Phase 1 is stirred with a Rayneri blender equipped with a deflocculating paddle, stored beforehand in an oven at 55° C. in order to avoid any phenomenon of recrystallization of the wax, and then the mixture is allowed to homogenize for a few seconds. Phase 1 is brought to a temperature of approximately 70° C. and then phase 2 is introduced into phase 1. The stirring speed is adjusted according to the consistency of the product. Where appropriate, the diethylene glycol monoethyl ether (“Transcutol P®”) is then immediately incorporated. The product obtained remains translucent until its temperature drops to approximately 45/50° C. Below this temperature, it begins to opacify and becomes white and more consistent. Phase 3 is then introduced at around 45° C. The stirring is maintained for a further 10 minutes, by varying the height of the paddle while at the same time allowing the mixture to gradually cool. When the temperature of the mixture is approximately 35° C., the stirring is stopped and the product obtained is packaged.

### EXAMPLE 6-STABILITY STUDY

The physical and chemical stability characteristics of the composition of example 3 described above are given below.

The study of the physical stability of the compositions is carried out by macroscopic observation, which makes it possible in particular to evaluate phase-separation phenomena, and by microscopic observation, which makes it possible in particular to evaluate active ingredient recrystallization phenomena.

This physical stability study is carried out for three months on the composition placed either at 4° C. or at ambient temperature or else at 40° C. The observations made at the moment of implementation, one month, two months and three months later, show no change in the appearance of the compositions, irrespective of their storage temperature. The composition of example 3 is therefore physically stable.

The chemical stability study is carried out by assaying the vitamin D derivative at the moment of implementation of the study (T0), and one month after (T1), two months after (T2) and, finally, three months after the beginning of implementation of the study (T3), on the composition of example 3 placed either at ambient temperature or at 40° C. The results are given in table 2 below.

In this table, the values presented without a unit represent the percentage by weight of vitamin D derivative assayed in the composition, expressed relative to the total weight of the composition.

The percentages presented reflect, for their part, the ratio of the percentage by weight measured in the composition, to the percentage by weight theoretically introduced (0.1%).

TABLE 2

| | |
|---|---|
| T0 | 0.099 (99.0%) |
| | CV = 0.9% |
| Stability at ambient temperature | |
| T1 | ND |
| T2 | 0.099 (99.1%) |
| | CV = 0.9% |
| T3 | 0.098 (98.3%) |
| | CV = 1.2% |
| Stability at +40° C. | |
| T1 | 0.098 (97.5%) |
| | CV = 1.5% |
| T2 | 0.099 (99.9%) |
| | CV = 1.3% |
| T3 | 0.096 (95.9%) |
| | CV = 2.0%) |

The content of vitamin D derivative was assayed by HPLC.

It is noted that the content of vitamin D derivative does not significantly vary over the period of the study at ambient temperature and at 40° C.

It therefore results from these observations that the composition of example 3 comprising 0.1% by weight of vitamin D derivative remains stable over time.

### EXAMPLES 7 AND 8 (COMPARATIVE)

### Comparative Study of Release-Penetration in the Skin of an Active Ingredient According to the Type of Composition Used

The aim of this study is to compare the in vitro release-penetration of the vitamin D derivative: (4E,6E)-7-[3-(3,4-bishydroxymethylbenzyloxy)phenyl]-3-ethylnona-4,6-dien-3-ol, contained at a content of 0.3% (weight/weight) when this active ingredient is formulated in a gel-type preparation in accordance with the invention, compared with that of the same active ingredient contained in the same proportion in an ointment-type reference preparation.

The compositions of the gel and of the ointment are given in table 3 below.

In this table, the amounts indicated are as a percentage by weight and are expressed relative to the total weight of the composition.

TABLE 3
TYPES OF COMPOSITION

| | | Example 7 | Example 8 |
|---|---|---|---|
| INGREDIENTS | FUNCTION | (gel) | (ointment) |
| Vitamin D derivatives: | Active | 0.30 | 0.30 |
| (4E,6E)-7-[3-(3,4- | ingredient | | |
| bishydroxymethylbenzyloxy)- | | | |
| phenyl]-3-ethylnona-4,6-dien- | | | |
| 3-ol | | | |
| Mixture of silicone elastomer | Gelling agent | 44.90 | — |
| and of | | | |
| decamethylcyclopentasiloxane | | | |
| (“ST-Elastomer 10 ®” DOW | | | |
| CORNING) | | | |
| Decamethylcyclopentasiloxane | Diluting agent | 26.80 | — |
| (“Mirasil CM5” from Rhodia) | | | |
| (C₁₆-C₁₈) atomized glyceryl | Occlusive | 8.00 | — |
| dipalmitostearate (“Preciro | thickening | | |
| l ATO 5 ®” from | agent | | |
| GATTEFOSSE) | | | |
| Isopropyl myristate | Emollient | 10.00 | — |
| Propylene glycol | Propenetrating | 5.00 | 10.00 |
| | agent | | |
| Paraffin oil | Emollient | — | 5.00 |
| Absolute ethanol | Solvent | 5.00 | — |
| Petroleum jelly | Occlusive | — | 76.94 |
| | thickening | | |
| | agent | | |
| Macrogol 2 stearyl ether | Emulsifier | — | 5.00 |
| Sodium edetate | Chelating agent | — | 0.0065 |
| Disodium phosphate dihydrate | Buffer | | 0.026 |
| DL-alpha-tocopherol | Antioxidant | — | 0.12 |
| Water | | — | qs for 100 |

Each preparation was applied, in vitro, to human skin of controlled thickness under nonocclusive conditions. Sixteen hours after application thereof, the distribution of the active ingredient was quantified in the various skin compartments, epidermis, stratum corneum, dermis and recipient liquid. In addition, the mass balance was determined for each of the preparations, taking into account the dose not absorbed. All the samples are analyzed by HPLC using a “Symmetry C8®”, 3.5 μm, 50×2.1 mm column, an aqueous-alcoholic mixture as mobile phase and with TIS/MS/MS detection.

More specifically, the study is carried out using Franz diffusion cells with a diffusion surface area of 2 cm². Abdominal samples of human skin of controlled thickness are taken from six different patients (5 women and 1 man) 37 to 72 years old. The dermal face of the skin is brought into contact with 3 ml of an isotonic solution with continuous agitation in the static mode, i.e. without renewal of the recipient liquid throughout the entire experiment, and under thermostatic conditions at 37° C. Each preparation is applied in duplicate to skin samples from three different donors (which therefore corresponds to six cells per preparation).

A target dose of 10 mg of preparation per square centimeter is applied to the surface of the skin, which corresponds precisely to a dose of 30 μg of active ingredient per square centimeter. The exposure time, i.e. the time elapsed from the application of the preparation to be tested until its removal by washing the skin, is 16 hours under nonocclusive conditions.

Sixteen hours after application, after standardized elimination of the surface excess, the distribution of the active ingredient is quantified in the various skin compartments and in the recipient liquid.

In addition, the mass balance is determined for each preparation, taking into account the dose not absorbed. All the samples are analyzed using a method of HPLC with TIS/MS/MS detection, the lower limit of quantification being 10 ng/ml.

As regards the experimental conditions, transepidermal water loss (TEWL) is used to evaluate the integrity of the stratum corneum. The mean TEWL rate measured is 4.1±0.6 g.m⁻².h⁻¹. However, 8 values out of 48 differ significantly from the baseline value.

As regards the thickness of the skin, despite a considerable variability between the various donors (from 0.83 to 1.85 mm), there is no significant variation between the average thickness of the skin used for each preparation.

The average mass balances are considered to be acceptable for nonradioactive test samples (greater than or equal to 84% of the dose applied).

As regards the contents of active ingredient recovered in the various skin compartments, the experimental results show that, irrespective of the preparation tested, the active ingredient is distributed in the skin (epidermis, stratum corneum included, and dermis). At the end of the exposure period (16 hours), the content of active ingredient in the sample originating from the recipient liquid is less than the limit of quantification. The distribution in the skin is different according to the preparation type: with the ointment-type preparation, the active ingredient is distributed identically in the dermis (stratum corneum included) and in the dermis, whereas, with the gel-type preparation, the active ingredient is mainly present in the epidermis (including the stratum corneum). The amount of active ingredient present in this compartment is 4 times greater than that obtained with the ointment. As regards the dermis, the amount of active ingredient obtained with the gel is equivalent to that obtained with the ointment.

The total amounts of active ingredient having penetrated into the skin, considered as a whole, and into the recipient liquid are:

Ointment:
0.63±0.14 μg (i.e. 2.3% of the dose applied) with a mass balance of 97±3%, and

Gel:
1.90±0.46 μg (i.e. 6.7% of the dose applied) with a mass balance of 84±4%.

It is therefore noted that, when the active ingredient is formulated in a preparation or composition in the form of a gel in accordance with the invention, its degree of penetration is three times higher than that obtained when the same active ingredient is formulated at the same dose by weight in an ointment-type preparation. Consequently, the compositions in accordance with the invention therefore make it possible to significantly increase the release-penetration of the active ingredients that they contain.

1-32. (canceled)

33. An anhydrous pharmaceutical composition combining at least one active ingredient and a silicone agent comprising at least one organopolysiloxane elastomer and a thickening agent different from the silicone agent, said active ingredient being in a solubilized form in said composition.

34. An anhydrous pharmaceutical composition combining at least one active ingredient and a silicone agent, said active ingredient being in a solubilized form in said composition, said silicone agent comprising at least one organopolysiloxane elastomer prepared by crosslinking reaction between:
polysiloxanes (A) containing ≡Si—H groups represented by the compounds of formula R¹⁴₃SiO(R¹⁵SiO)ₐ(R¹⁶HSiO)_{b}SiR¹⁴₃ here denoted as type A¹, and compounds of formula HR¹⁴₂SiO(R¹⁵₂SiO)_{c}SiR¹⁴₂H or of formula HRZ¹⁴₂SiO(R¹⁵₂SiO)ₐ(R¹⁶HSiO)_{b}SiR¹⁴₂H here denoted as type A², in these formulae, R¹⁴, R¹⁵ and R¹⁶ are alkyl groups having from 1 to 6 carbon atoms, a is an integer ranging from 0 to 250, b is in integer ranging from 1 to 250, and c is an integer ranging from 0 to 250, the molar ratio of the compounds A²: A¹ being from 0 to 20,
an alpha, omega-diene (B) which is a compound of formula CH₂═CH(CH₂)_{d}CH═CH₂ in which d is an integer ranging from 1 to 20, in the presence of a catalyst, and
a low molecular weight linear or cyclic polysiloxane (C), being a cyclic volatile methylsiloxane chosen among hexamethylcyclotrisiloxane, octamethylcyclotetrasiloxane, decamethylcyclopentasiloxane and dodecamethylcyclohexasiloxane.

35. The composition as claimed in claim 33, wherein it is intended for topical application.

36. The composition as claimed in claim 33, wherein it is in the form of a gel.

37. The composition as claimed in claim 33, wherein it has a water content of less than or equal to 5%, in particular less than or equal to 3% by weight relative to the total weight of the composition, and in particular equal to zero.

38. The composition as claimed in claim 33, wherein said active ingredient is solubilized using at least one solvent agent.

39. The composition as claimed in claim 33, wherein it comprises at least one silicone agent, a hydrocarbon-based compound, in particular of pasty or solid type, an active ingredient in a solubilized form, and an alcohol-type solvent.

40. The composition as claimed in claim 33, characterized in that said active ingredient is chosen from vitamin D and its derivatives.

41. The composition as claimed in claim 33, wherein said active ingredient is present at a content of from 0.0001 to 20%, in particular from 0.01 to 15%, and more particularly from 0.025 to 5% by weight relative to the total weight of the composition.

42. The composition as claimed in claim 38, wherein said solvent agent is chosen from alcohols such as ethanol.

43. The composition as claimed in claim 38, wherein the content of solvent agent is from 1% to 50%, in particular from 2% to 40%, and more particularly from 5% to 10% by weight relative to the total weight of the composition.

44. The composition as claimed in claim 33, wherein the content of elastomer is from 1% to 20%, and in particular from 4% to 12% by weight relative to the total weight of the composition.

45. The composition as claimed in claim 33, wherein the elastomer is formulated in at least one volatile silicone oil.

46. The composition as claimed in claim 45, wherein said volatile silicone oil is chosen from linear or cyclic polyorganosiloxane oils having from 2 to 10 silicon atoms.

47. The composition as claimed in claim 33, wherein the content of silicone agent is from 20% to 80%, and in particular from 30% to 70% by weight relative to the total weight of the composition.

48. The composition as claimed in claim 33, wherein said thickening agent is chosen from solid or pasty hydrocarbon-based compounds.

49. The composition as claimed in claim 39, wherein the hydrocarbon-based compound is chosen from hydrocarbon-based waxes of animal, plant, mineral or synthetic origin, and mixtures thereof.

50. The composition as claimed in claim 49, wherein said hydrocarbon-based wax is chosen from glyceryl esters and esters of saturated, unsaturated and in particular polyunsaturated fatty acids having from 10 to 24 carbon atoms.

51. The composition as claimed in claim 50, wherein said wax is chosen from glyceryl behenate and glyceryl dipalmitostearate, and mixtures thereof.

52. The composition as claimed in claim 39, wherein said hydrocarbon-based compound is present at a content ranging from 2% to 80%, and in particular from 4% to 30% by weight relative to the total weight of the composition.

53. The composition as claimed in claim 39, wherein said composition has occlusive properties.

54. The composition as claimed in claim 33, wherein it also comprises at least one agent for diluting the silicone agent.

55. The composition as claimed in claim 54, wherein said diluting agent is chosen from linear or cyclic volatile silicone oils.

56. The composition as claimed in claim 33, wherein it also comprises at least one agent for promoting the penetration of the active ingredient into the skin.

57. The composition as claimed in claim 56, wherein said agent for promoting the penetration of the active ingredient into the skin is chosen from glycols, glycol ethers, fatty acids, fatty acid esters, glycol esters, glycerides, azones, polysorbates, alkanols, dimethyl sulfoxide, and mixtures thereof.

58. The composition as claimed in claim 56, wherein said agent for promoting the penetration of the active ingredient into the skin is present at a content ranging from 2% to 30%, and in particular from 4% to 25% by weight relative to the total weight of the composition.

59. The composition as claimed in claim 33, wherein it also comprises at least one pharmaceutically acceptable additive.

60. A method for preparation of an anhydrous pharmaceutical composition comprising at least one active ingredient in a solubilized form and with prolonged stability, comprising combining said active ingredient, at least one organopolysiloxane elastomer and a thickening agent different from said silicone agent.

61. A method for the preparation of an anhydrous pharmaceutical composition comprising at least one active ingredient in a solubilized form and with prolonged stability comprising combining said active ingredient and a silicone agent as defined in claim 34.

62. The method as claimed in claim 60 wherein said silicon agent is the content of elastomer is from 1% to 20%, and in particular from 4% to 12% by weight relative to the total weight of the composition..

63. The use as claimed in claim 60 wherein said composition is an anhydrous pharmaceutical composition combining at least one active ingredient and a silicone agent comprising at least one organopolysiloxane elastomer and a thickening agent different from the silicone agent, said active ingredient being in a solubilized form in said composition.

64. A method for the treatment of psoriasis, comprising administering to a subject requiring treatment of psoriasis a composition as claimed in claim 33.
